(19) United States
(12) Patent Application Publication
(10) Pub. No.: US 2011/0008299 A1
(43) Pub. Date: Jan. 13, 2011

(54) TREATMENT OF PELVIC FLOOR DISORDERS WITH AN ADIPOSE-DERIVED CELL COMPOSITION

(75) Inventors: Edouard A. Koullick, Golden Valley, MN (US); Tania M. Schroeder, Hastings, MN (US); Natalie Ann Borgos, Lino Lakes, MN (US)

Correspondence Address: AMS RESEARCH CORPORATION, 10700 BREN ROAD WEST, MINNETONKA, MN, 55343, US

(73) Assignee: AMS RESEARCH CORPORATION, Minnetonka, MN (US)

(21) Appl. No.: 12/920,570

(22) PCT Filed: Mar. 26, 2009
(86) PCT No.: PCT/US09/38426

§ 371 Date: Sep. 1, 2010

Related U.S. Application Data
(60) Provisional application No. 61/039,652, filed on Mar. 26, 2008.

(51) Int. Cl. A61K 35/12; A61P 15/00; A61P 13/10

(52) U.S. Cl. 424/93.7

(57) ABSTRACT

A method for treating a pelvic floor disease comprises removing adipose tissue from a patient, processing a first portion of the adipose tissue to obtain a heterogeneous mixture of cells that includes adipose-derived stem cells, combining the heterogeneous mixture of cells with a second, unprocessed portion of the adipose tissue in a ratio of from approximately 1:1 to 1:4 to produce a cell composition, wherein the second portion of the adipose tissue is structured to provide a natural scaffold, and administering the cell composition to the patient to treat a pelvic floor disease.

### FIELD OF THE INVENTION

The invention relates generally to cells derived from adipose tissue. More particularly, the invention relates to preparing and using adipose-derived cells compositions to treat pelvic floor disease.

### BACKGROUND OF THE INVENTION

The concept of utilizing stem cells for regenerative medicine purposes has advanced considerably in recent years. The advancements have resulted at least in part by the discovery and identification of stem cells in various tissues. Although regenerative therapy has not yet reached a point where it is conventionally applied to numerous patients suffering from organ or tissue dysfunction, there is an increasing demand for therapies using stem cells as a form of regenerative medicine.

Mesenchymal cells, such as adipocytes, bone cells, ligament cells, cardiac muscle cells, and the like, have an important function of forming the shape or skeleton of the body. Therefore, there is an increasing expectation for the application of groups of such cells or tissues of such cells to regenerative medicine and implantation medicine. Mesenchymal stem cells are a type of tissue stem cells. Mesenchymal stem cells naturally occur only in a small amount (about one ten thousandth of all cells in the bone marrow). As a result, it is difficult to isolate mesenchymal stem cells. Furthermore, harvesting such cells from bone marrow is generally associated with a lot of pain to the donor.

More recently, it has been found that stem cells may also be harvested from fat tissue. Particularly, a large amount of stem cells can be obtained from fat as compared to other tissues, such as bone marrow. Furthermore, the density of these stem cells from fat tissue has also been found to be much greater. For this reason, the use of fat tissue as a source of stem cells has drawn a lot of attention. However, minimal focus has been placed on harvesting stem cells from fat tissue for use in pelvic disorders such as, for example, urinary incontinence, prolapse, fecal incontinence, erectile dysfunction, and interstitial cystitis.

Urinary Incontinence

In the United States and Europe, urinary incontinence is believed to affect over fifty million women and over 800,000 men. More than 600,000 surgeries are performed on women and more than 10,000 surgeries are performed on men each year to address urinary incontinence. The social implications for an incontinent patient include loss of self-esteem, embarrassment, restriction of social and sexual activities, isolation, depression and, in some instances, dependence on caregivers. Incontinence is believed to be one of the most common reasons for institutionalization of the elderly.

There are five basic types of incontinence: stress incontinence, urge incontinence, mixed incontinence, overflow incontinence and functional incontinence. Stress urinary incontinence (“SUI”) is the involuntary loss of urine that occurs due to sudden increases in intra-abdominal pressure resulting from activities such as coughing, sneezing, lifting, straining, exercise and, in severe cases, even simply changing body position. Urge incontinence, also termed “hyperactive bladder,” “frequency/urgency syndrome” or “irritable bladder,” occurs when an individual experiences the immediate need to urinate and loses bladder control before reaching the toilet. Urge urinary incontinence is thought to involve overactivity in the detrusor muscle (which contracts to expel urine from the bladder) and leads to a number of symptoms including urge sensation, increased urinary frequency, and nocturia. Detrusor overactivity may result from interference with normal neurological function or from defects in detrusor muscle cells that result in hypersensitivity to excitatory stimuli. Mixed incontinence is a combination of the symptoms for both stress and urge incontinence and is the most common form of urinary incontinence. Overflow incontinence is a constant dripping or leakage of urine caused by an overfilled bladder. This form of incontinence accounts for approximately 10-15% of incontinence cases and is often caused by a blockage or obstruction of the outlet from the bladder (such as from an enlarged prostate). Functional incontinence results when a person has difficulty moving from one place to another. It is generally caused by factors outside the lower urinary tract, such as deficits in physical function and/or cognitive function and accounts for about one quarter of incontinence cases.

A variety of treatment options are currently available to treat incontinence. Some of these treatment options include external devices, behavioral therapy (such as biofeedback, electrical stimulation, or Kegel exercises), injectable materials for bulking the bladder sphincter or periurethral tissues, prosthetic devices to control urine flow (such as artificial sphincters) and surgery. Depending on age, medical condition, and personal preference, surgical procedures can be used to completely restore continence.

One type of procedure, found to be an especially successful treatment option for SUI in both men and women, is a sling procedure. A sling procedure is a surgical method involving the placement of a sling to stabilize or support the bladder neck or urethra. There are a variety of different sling procedures. Slings used for pubovaginal procedures differ in the type of material and anchoring methods. In some cases, the sling is placed under the bladder neck and secured via suspension sutures to a point of attachment (e.g. bone) through an abdominal and/or vaginal incision.

Another procedure, the TVT Tension-free Vaginal Tape procedure, utilizes a Prolene™ nonabsorbable, polypropylene mesh. The mesh is a substantially flat, rectangular knitted article. The mesh includes a plurality of holes that are sized to allow tissue ingrowth to help avoid infection. A plastic sheath surrounds the mesh and is used to insert the mesh. During the sling procedure, incisions are made in the abdominal (i.e. suprapubic) area and in the vaginal wall. Two curved, needle-like elements are each connected to an end of the vaginal sling mesh. A sling-free end of one of the needle-like elements is initially pushed through the vaginal incision and into the periurethral space. Using a handle attached to the needle, the needle is angulated laterally (for example, to the right) to perforate the endopelvic fascia, guided through the retropubic space and passed through the abdominal incision. The handle is disconnected and the needle is then withdrawn through the abdominal wall, thereby threading a portion of the sling through the tissue of the patient. The handle is then connected to the other needle and the technique is repeated on the contralateral side, so that the mesh is looped beneath the bladder neck or urethra. The sling is positioned to provide appropriate support to the bladder neck or urethra. Typically a Mayo scissors or blunt clamp is placed between the urethra and the sling to ensure ample looseness of the sling. When the TVT mesh is properly positioned, the cross section of the mesh should be substantially flat. In this condition, the edges of the mesh do not significantly damage tissue. The sling ends are then cut at the abdominal wall, the sheath is removed and all incisions are closed. Also, an artificial sphincter may be introduced surgically to gain control over urinary emissions.

In addition to surgical procedures that alter positions of the bladder or bladder neck, bulking agents may be injected either directly into the sphincter or into spaces around the urethra. These bulking agents are believed to increase resistance to the flow of urine into and through the urethra giving the patient greater control over urinary emissions. A number of agents have been employed as periurethral bulking agents including cross-linked collagen, carbon coated beads and a biocompatible copolymer implant (e.g., Tegress™ Urethral Implant). Re-absorption by the body can limit long term effectiveness of this approach, especially for cross-linked collagen.

Autologous chondrocytes, autologous skeletal and smooth muscle, along with autologous fat are other implant materials that have been investigated. Injection of autologous fat (adipose tissue) may provide relief from symptoms of SUI, but the tissue is often resorbed by the body thereby providing only short term relief. Treatments involving injection of chondrocytes and autologous smooth muscle cell treatments are also believed to be short lived in effectiveness. Moreover, use of these cells requires biopsy and extended periods of cell culture under carefully controlled conditions to expand cell populations to the point of having enough cells to inject. Skeletal muscle cells have also been used for injection into the bladder sphincter and to periurethral regions. The approaches that have been described for use of skeletal muscle similarly require cell culture techniques to select cell subpopulations from a biopsy for injection and may require expansion of those cell subpopulations in extended culture to obtain sufficient cellular material for injection.

Thus, there is a desire to obtain a minimally invasive yet effective surgical procedure to treat incontinence, specifically stress urinary incontinence, that can be used with minimal to no side effects. Such a procedure should reduce the complexity of current procedures.

Prolapse

Pelvic organ prolapse is defined as the decent of one or more abdominal organs (including the small bowel, uterus, bladder, rectum, urethra, and vagina) from a normal abdominal location. Prolapse involving the small bowel or uterus may lead to prolapse of the vagina, even to the point of eversion from the body. Prolapse may lead to varying degrees of discomfort in patients, to incontinence of varying severity and to other effects including painful intercourse. It is estimated that seven million women may have severe prolapse and over 600,000 surgeries are performed in the United States and Europe to address the sequellae of prolapse.

Prolapse is thought to be caused by injury to anatomic supports that normally hold the pelvic organs in place or by other dysfunction that allows the pelvic organs to descend. A number of connective tissues, including the endopelvic fascia, vesicovaginal adventitia, pubocervical fascia and rectovaginal fascia all provide support to abdominal organs.

Damage to connective tissue, muscle and nerves innervating muscle attached to pelvic organs, directly or indirectly, is thought to account for a significant portion of prolapse cases. This damage may come from repeated exertion of muscles over time, such as during pregnancy, from repeated heavy lifting or even from chronic coughing. Damage to connective tissue may also come from less frequent, but more traumatic events, such as birth by vaginal delivery or hysterectomy.

Selection of surgical treatment for a prolapse condition is governed in large part by the organs affected, as well as by the severity of the condition, the involvement of other organs and potentially the existence of other medical conditions. Surgery is frequently effective in restoring the affected pelvic organs to their appropriate position. It is recognized, however, that surgical procedures to correct prolapse involving one set of organs, may lead to prolapse involving other organs.

Thus, there is a desire to obtain a minimally invasive yet effective surgical procedure to treat pelvic organ prolapse that can be used with minimal to no side effects. Such a procedure should reduce the complexity of current procedures.

Fecal Incontinence

Fecal incontinence may result from a number of causes and many may suffer transient fecal incontinence simply as a result of loose stool or diarrhea. On the other hand, constipation can also lead to fecal incontinence when watery stool leaks around impacted stool and past anal sphincters stretched by the stool. Longer term fecal incontinence may result from pelvic floor disorders, including herniation of the rectum into the vagina or rectal prolapse. Nerve damage affecting sensory or motor control in the anal sphincter muscles may also lead to fecal incontinence.

Such damage may arise during surgery or from traumatic injury. Damage to the anal sphincter muscles themselves can lead to loss of control over the contraction of the sphincters, leading to incontinence. One of the major causes of damage to anal sphincter muscle results from vaginal delivery of children. More than five million people in the United States are believed to be affected by fecal incontinence.

Treatments for fecal incontinence include diet changes (including addition of fiber to the diet) and bowel training systems to achieve regularity. Medications, such as antidiarrheal medications can give patients more control over bowel movements by controlling rectal contractions or by providing additional consistency to the stool. A number of different surgeries may be used to address fecal incontinence, depending on the cause and severity of the problem. In sphincteroplasty, a sphincter is cut in the region of a defect or injury and the two ends are overlapped and then sewn in place. In other cases, muscle transposition is used to repair the sphincter by surrounding the anal canal with skeletal muscle (from forearm, thigh or buttock) to allow for restoration of voluntary control. Artificial sphincters may also be used to provide assurance of control over passing of stool. Protocols for injection of bulking agents into the anal sphincter or the regions surrounding the anal sphincter are receiving increased attention; however the use of these agents is still limited.

Thus, there is a desire to obtain a minimally invasive yet effective surgical procedure to treat fecal incontinence that can be used with minimal to no side effects. Such a procedure should reduce the complexity of current procedures.

Erectile Dysfunction

Erectile dysfunction is believed to affect more than ninety million men in the United States and Europe, with seventeen million presenting with severe conditions that greatly interfere with the ability to initiate and maintain erections. Erectile dysfunction may arise from a number of causes. Age brings on a lack of arterial elasticity in vessels supplying blood to erectile tissues. Damage to nerves necessary for initiating and sustaining erections brought on by chronic conditions (such as diabetes) or by injury can lead to dysfunction. A significant cause of nerve damage comes from injury that occurs during prostate surgeries, especially radical prostatectomies. Although new surgical procedures have been introduced that conserve the nerves in this region, a majority of men who undergo prostate surgery can still expect some degree of post operative erectile dysfunction.

The penis is comprised of three erectile bodies, which include two parallel bodies termed corpus cavernosa and another body termed the corpus spongiosum positioned underneath and wedged in between the corpus cavernosas. The three erectile bodies are heavily vascularized and contain large proportion of smooth muscle cells. Erection is caused by neurologically-induced relaxation of smooth muscle cells in the erectile bodies, which allows influx and accumulation of blood into the balloon-like sacs between the smooth muscle cells called sinusoids. As blood accumulates, the outflow of blood is prevented by pressure from the tunica albuginea against the venous plexus, thus causing trapping of the blood, allowing erection to occur. The process of blood accumulation due to venous trapping is termed the veno-occlusive mechanism. Additional rigidity of the penis shaft is provided by contraction of the ischiocaverous muscles.

It is known that erectile dysfunction is multifactorial, with causative influences including vasculogenic, endocrinological, psychogenic, and neurogenic. Some studies have found that vascular disease may be the direct or indirect culprit in many cases of erectile dysfunction. Vascular disease is associated with either decreased production of nitric oxide (NO), or decreased responsiveness to its actions. There are five general mechanisms postulated for decrease in this intermediate, all five of which have been found to be associated with erectile dysfunction.

The first mechanism is oxidative stress in the form of the oxygen free radical superoxide, which both enhances degradation of NO (by direct conversion to peroxynitrite), as well as decreases its synthesis. The second mechanism is preformed advanced glycation end products, which are found in diabetics, as well as at higher concentrations in elderly patients. These inactivate NO may directly induce an increased production of superoxide, which may also inhibit NO as previously mentioned, and may directly suppress synthesis of endothelial nitric oxide synthase (eNOS) by endothelial cells. The third mechanism is enhanced expression of the enzyme arginase II, which compete with nitric oxide synthase for arginine. Thus enhanced arginase expression, which is associated with ED, leads to inhibition of NO. The forth mechanism is reduced transcription of eNOS and nNOS in tissue lacking testosterone. Finally, the fifth mechanism is increased activity of the Rho/Rho kinase which is associated with atherosclerosis. The Rho/Rho kinase has been found to both inhibit NOS activity and increase vascular smooth muscle tone, thereby inhibiting NO and erectile ability, respectively. In all of the above five mechanisms, the primary cause may be endothelial dysfunction, which may be induced by aging, artherosclerotic changes, and oxidative stress.

A number of oral medications for treating erectile dysfunction have entered the marketplace in recent years, including VIAGRA®, CIALIS® and LEVITRA®. These medications all provide significant relief to a large segment of men with erectile dysfunction. However, they each require that the medication be taken in advance of initiation of sexual activity and their effects may be delayed if ingested with food.

Various treatments have also been tried in connection with erectile dysfunction, including administration of Prostaglandin E1 by injection into the cavemosum of the penis, by administration of a suppository into the urethra and by topical administration. These approaches allow for less advance preparation, but are not highly effective across patient populations, especially radical prostatectomy patients.

Surgical interventions are also available for addressing erectile dysfunction, especially where medications are ineffective or contraindicated. Penile implants of many different configurations are used to provide support for an erection. These implants are effective in restoring patient sexual satisfaction. Increasingly, these implants have been engineered to be completely concealed within the patient. However, implants may fail over time and replacement or total removal may be required potentially leaving the patient with no relief at all.

Thus, there is a desire to obtain a minimally invasive yet effective surgical procedure to treat erectile dysfunction that can be used with minimal to no side effects. Such a procedure should reduce the complexity of current procedures.

Interstitial Cystitis

Interstitial cystitis is a progressive syndrome affecting the urinary bladder and may present in ulcerative (or classic) or nonulcerative forms. Symptoms associated with interstitial cystitis include increased urgency and frequency of urination, as well as pelvic pain. Patients afflicted with interstitial cystitis also complain of more generalized symptoms that affect quality of life, often significantly, including chronic abdominal pain. Origin of this syndrome in patients is not well understood. While evidence of increased immune function in the region of bladder muscle has been observed in patients (typically higher numbers of immune system cells), no bacterial or other agents have been consistently associated with this syndrome.

Numerous oral agents have been tested for treatment of interstitial cystitis. These agents include L-arginine, pentosan polysodium sodium, cimetidine, gabapentin, suplatast tosilate (an immunoregulator), quercetin, Nerve Growth Factor (NGF) and montelukast (a leukotriene receptor antagonist). Intravesical treatments have also been evaluated. Lidocaine, heparin, BCG, hyaluronic acid and vanilloids have shown varying degrees of success in relieving symptoms. Interventional treatments, such as sacral neuromodulation have been tried, but these are costly in the long term and invasive. Surgical treatment for interstitial cystitis may involve ablation procedures or in severe cases, removal of the bladder. This radical approach is very often successful in alleviating symptoms if interstitial cystitis. However, patients will likely desire a bladder substitute to maintain as normal a lifestyle as possible, thereby requiring additional surgery.

Thus, there is a desire to obtain a minimally invasive yet effective surgical procedure to treat interstitial cystitis, and that can be used with minimal to no side effects. Such a procedure should reduce the complexity of current procedures.

For purposes of background and understanding, several terms related to the use of adipose-derived cells for therapeutic purposes will be discussed to follow. The descriptions of the terms are not intended to define or limit their meaning, but rather to provide a general understanding of what may be encompassed by the use of the terms.

As used herein, “adipose tissue” generally refers to a tissue containing multiple cell types including adipocytes and microvascular cells. As appreciated by those skilled in the art, adipose tissue may include stem cells and endothelial precursor cells. Accordingly, adipose tissue refers to fat including the connective tissue that stores the fat.

The term “cell” is used herein in its broadest sense in the art, referring to a structural unit of tissue of a multicellular organism, which is capable of self replicating, has genetic information and a mechanism for expressing it, and is surrounded by a membrane structure which isolates the living body such as a cell from the outside.

Fat or adipose cells (“adipocytes”) and their corresponding material may be derived from any organism (e.g., Myxiniformes, Petronyzoniformes, Chondrichthyes, Osteichthyes, Amphibia, Reptilia, Aves, Mammalia, etc.), more preferably mammalian (e.g., Monotremata, Marsupialia, Edentate, Dermoptera, Chiroptera, Carnivora, Insectivora, Proboscidea, Perissodactyla, Artiodactyla, Tubulidentata, Pholidota, Sirenia, Cetacean, Primates, Rodentia, Lagomorpha, etc.) as long as such an organism has adipocytes or cells corresponding thereto. In one exemplary embodiment, the cells may be derived from a human.

As used herein, the term “stem cell” may refer to a multipotent cell with the potential to differentiate into a variety of other cell types, which perform one or more specific functions and have the ability to self-renew. Some stem cells may be pluripotent.

As used herein, “precursor cell” refers to a cell which corresponds to an undifferentiated parent cell having no differentiation property, when the progeny cell thereof is known to have a specific differentiation property, and includes not only multipotent undifferentiated cells but also monopotent undifferentiated cells. For example, when a progeny cell is a vascular endothelial cell, then the precursor cell thereof is a vascular endothelial precursor cell. As used herein, the term “stem cell” may encompass precursor cells. However, it can be said that a precursor cell obtained by differentiation of a stem cell corresponds to a “differentiated cell” in terms of the stem cell.

The term “processed lipoaspirate” (PLA) may refer to a precursor cell which is obtained from the fat portion (lipoaspirate) of an aspirate from liposuction. PLA may also refer to adipose tissue that has been processed to separate the active cellular component (e.g., the component containing stem cells) from the mature adipocytes and connective tissue. For example, PLA may refer to the plurality of cells obtained by washing and separating the cells from the adipose tissue.

The term “adipose-derived precursor cell” may refer to a stem cell and also other precursor cells, such as stem cells from peripheral blood or vascular-stromal cells (preadipocytes), obtained from liposuction. Adipose-derived precursor cells may include any multipotent or monopotent precursor cell populations derived from the adipose tissue or obtained from a liposuction procedure. The cells may include adipose-derived vascular-stromal cells (preadipocytes, adipose-derived interstitial cells), adipose-derived stem cells, fat stem cells, endothelial progenitor cells, hematopoietic stem cells, and so on.

The term “mesenchymal stem cell” (MSC) may refer to a stem cell found in mesenchyme. Mesenchyme refers to a population of free cells which are in an asterodal shape or have irregular projections and bridge gaps between epithelial tissues, and which are recognized in each stage of development of multicellular animals. Mesenchyme may also refer to tissue formed with intracellular cement associated with the cells. Mesenchymal stem cells typically have the ability to differentiate into numerous types of cells including bone cells, chondrocytes, muscle cells, stroma cells, tendon cells, and adipocytes.

The term “adipocyte” may refer to a cell which is located between tissues or forms fat tissue as areolar tissue or a group along capillary blood vessels, and which contains a large amount of lipid. Fat cells may include a yellow adipocyte and a brown adipocyte.

The term “autologous” or “self” in relation to an entity refers to the whole or a part (e.g., a cell, a tissue, an organ, etc.) of the same entity. As used herein, the term “autologous” or “self” may encompass a graft from a genetically identical individual (e.g. an identical twin) in a broad sense.

### BRIEF SUMMARY OF THE INVENTION

The present invention encompasses adipose-derived stem cells and compositions thereof that may be inserted into a body in order to treat various conditions. In one aspect, the present invention provides a heterogeneous mixture of adipose-derived cells that may be employed, alone or within biologically-compatible compositions, to generate differentiated tissues and structures. Considering how plentiful adipose tissue is, the adipose-derived cells represent a ready source of pluripotent stem cells and other types of precursor and mesenchymal cells. Compositions that are administered to a patient include a mixture of adipose tissue and stem cells so that the composition has a higher concentration of stem cells than when the adipose tissue was removed from the patient.

In accordance with the present invention, a heterogeneous mixture of cells that includes adipose-derived stem cells may be obtained from adipose tissue via any suitable method. However, a first step in any such method requires the isolation of adipose tissue from the source. For example, human adipose stromal cells may be obtained from living donors using well-recognized protocols such as surgical or suction lipectomy. However derived, the adipose tissue is processed to separate the heterogeneous mixture of cells from the remainder of the material. In one protocol, the adipose tissue may be washed with physiologically-compatible saline solution and then vigorously agitated and left to settle, or alternatively centrifuged, a step that removes loose matter (e.g., damaged tissue, blood, erythrocytes, etc.) from the adipose tissue.

In one aspect of the present invention, raw adipose tissue removed from a recipient's body may be processed to substantially remove mature adipocytes and connective tissue thereby obtaining a heterogeneous mixture of cells that includes adipose-derived stem cells and that is suitable for administration into the recipient's body. In one embodiment the adipose-derived cells may be administered to the recipient's via injection with any prior mixing. In another embodiment the adipose-derived cells may be injected into the recipient in combination with other cells, tissue, tissue fragments, or other stimulators of cell growth and/or differentiation. In one exemplary embodiment, the adipose-derived cells, along with any of the above mentioned additives, are injected or otherwise surgically placed into the person from whom they were obtained in the context of a single operative procedure with the intention of deriving a therapeutic or structural benefit to the recipient.

In one exemplary embodiment in accordance with the present invention, a method of treating a patient may include the steps of: (i) removing adipose tissue from a patient; (ii) processing at least a portion of the removed adipose tissue to obtain a concentration of stem cells derived from the adipose tissue, said processing including washing the adipose tissue, centrifuging the adipose tissue, enzymatically dissociating the adipose tissue using trypsin, collagenase, dispase or combinations thereof to produce a heterogeneous concentrated stem cell population including myoblasts, fibroblasts, nerve cells, endothelial cells and adipocytes; (iii) washing the concentrated stem cell population in a sterile balanced salt solution; (iv) centrifuging the concentrated stem cell population; (v) combining the concentrated stem cell population with unprocessed, autologous adipose tissue; and (iv) administering the stem cell and unprocessed, autologous adipose tissue composition to a patient.

In another exemplary embodiment in accordance with the present invention, a method of treating a patient may include the steps of: (i) providing an adipose tissue removal system; (ii) removing adipose tissue from a patient using the adipose tissue removal system, the adipose tissue having a concentration of heterogeneous cells; (iii) processing the adipose tissue to increase the concentration of stem cells in the adipose tissue; (iv) mixing the heterogeneous population of concentrated adipose-derived tissue with another portion of adipose tissue to create a composition; and (v) administering the composition to the patient.

The novel composition in accordance with the present invention includes a concentrated, heterogeneous cell population derived from a first portion of adipose tissue removed from a patient, and a second portion of adipose tissue removed from the patient, the resultant composition having a concentration of cells greater than either said first or second portion alone.

### DETAILED DESCRIPTION OF THE INVENTION

The present invention is directed to a cell population present in adipose tissue, and systems and methods for administering the cell population into a human or animal patient. The cell population of the adipose tissue may be used as a source of cells for therapeutic applications and the like. Among other things, the cells may be used for regenerative medicine, such as for diseases that can be treated with regenerating cells. The cells of the population may be administered to a patient without other adipocytes or connective tissue, or may be administered mixed together with adipose tissue or another biologically compatible material in a concentrated amount, as discussed herein.

Although the following disclosure will refer to specific embodiments, those skilled in the art will appreciate that these embodiments are being presented merely for purposes of example and not limitation. Thus, the intent of the following detailed description, although discussing exemplary embodiments, is to be construed to cover all modifications, alternatives, and equivalents of the embodiments as may fall within the spirit and scope of the invention as defined by the appended claims. As will become obvious to those skilled in the art, the present invention may be practiced in conjunction with various cell or tissue separation techniques that are conventionally used in the art. Therefore, only a limited number of the commonly practiced process steps are described herein that are sufficient to provide an understanding of the present invention.

It has been discovered that adipose tissue is an especially rich source of stem cells. This finding may be due, at least in part, to the ease of removal of the major non-stem cell component of adipose tissue, the adipocyte. For example, PLA may generally include stem cells at a frequency of 0.1% or more. As appreciated by those skilled in the art, the PLA may be a heterogeneous mixture of cells known as the stromal vascular fraction, of which adipose-derived stem cells are only one component. Particularly, the stromal vascular fraction generally comprises a mixture of cells that consists of adipose-derived stem cells, endothelial cells and their precursors, smooth muscle cells and their precursors, and various other types of cells.

Because adipose-derived stem cells are pluripotent cells, they are ideal as a cell source in the field of regenerative medicine. Particularly, such cells generally have the capacity to develop into mesodermal tissues, such as mature adipose tissue, bone, various tissues of the heart (e.g., pericardium, epicardium, epimyocardium, myocardium, pericardium, valve tissue, etc.), dermal connective tissue, hemangial tissues (e.g., corpuscles, endocardium, vascular epithelium, etc.), muscle tissues (including skeletal muscles, cardiac muscles, smooth muscles, etc.), urogenital tissues (e.g., kidney, pronephros, meta- and meso-nephric ducts, metanephric diverticulum, ureters, renal pelvis, collecting tubules, epithelium of the female reproductive structures), pleural and peritoneal tissues, viscera, mesodermal glandular tissues (e.g., adrenal cortex tissues), and stromal tissues (e.g., bone marrow). Of course, inasmuch as the cell can retain potential to develop into mature cells, it also can realize its developmental phenotypic potential by differentiating into an appropriate precursor cell (e.g., a preadipocyte, a premyocyte, a preosteocyte, etc.).

Adipose tissue may be removed from a body via a wide range of tissue removal techniques known to a person of ordinary skill in the art. For example, adipose tissue may be removed from a patient by suction-assisted lipoplasty, ultrasound-assisted lipoplasty, and excisional lipectomy, or a combination of such procedures. Because at least a portion of the tissue may be reimplanted into the patient to achieve the therapeutic or regenerative effect in accordance with the present invention, the tissue extraction may preferably be performed in a sterile or aseptic manner to minimize contamination. In one exemplary embodiment, suction assisted lipoplasty may be desirable to remove the adipose tissue from a patient as it provides a minimally invasive method of collecting tissue with minimal potential for cell damage that may be associated with other techniques, such as ultrasound assisted lipoplasty.

In accordance with the present invention, the adipose tissue that is removed from a patient may be collected into a device for further processing. The device may be designed for the purpose of collecting and processing adipose tissue in order to produce a heterogeneous mixture of cells such as the stromal vascular fraction previously described. Particularly, the adipose tissue that is removed from the patient may be processed to change the concentration of the cells in the heterogeneous mixture, including the adipose-derived stem cells. For example, in one exemplary embodiment of the present invention, patients receive a higher concentration of stem cells than the concentration of stem cells typically present in adipose tissue transplants and other similar stem cell based therapies. The concentrated cells may be administered in a composition that comprises adipose-derived stem cells and other stromal vascular fraction cells and that is substantially free from mature adipocytes and connective tissue. Alternatively, the concentrated cells may be administered in a composition comprising a portion of adipose tissue with an increased amount of stem cells. Thus, a composition in accordance with the present invention includes a concentration of stem cells that is greater than the concentration of stem cells found in an equivalent portion of non-processed adipose tissue. For example, the heterogeneous mixture of cells may contain about 1-2% stem cells. However, heterogeneous mixtures having higher and lower concentrations of stem cells are contemplated and within the intended scope of the present invention.

Preparation of the adipose-derived cell population may require depletion of the mature fat-laden adipocyte component of adipose tissue. As will be appreciated by those skilled in the art, this may typically be achieved by one or more washing and disaggregation steps in which the adipose tissue is first rinsed to reduce the presence of free lipids and peripheral blood elements, and then disaggregated to free intact adipocytes and other cell populations from the connective tissue matrix. In certain embodiments, the entire adipocyte component (i.e., the non-stem cell component) may be separated from the stem cell component of the adipose tissue. In other embodiments, only a portion of the adipocyte component is separated from the stem cells, thus producing a heterogeneous mixture as previously described that may include endothelial cells (and their precursors) and smooth muscle cells (and their precursors).

More particularly, in the washing/rinsing step the tissue may be mixed with one or more solutions to wash off free lipid and single cell components, such as those components found in blood, leaving behind intact adipose tissue fragments. For example, according to one exemplary rinsing step, the adipose tissue that is removed from the patient may be mixed with a saline or other physiologic solution. However, when a rinsing step is utilized, the intact adipose tissue fragments may be separated from the free lipid and cells by any other suitable means as will be appreciated by those skilled in the art including, but not limited to, centrifugation, filtration, and the like.

Once the tissue is rinsed, the intact tissue fragments may then be disaggregated using any suitable techniques or methods, including mechanical force, enzymatic digestion, or a combination of mechanical and enzymatic methods. For example, the cellular component of the intact tissue fragments may be disaggregated by methods using collagenase-mediated dissociation of adipose tissue, which are similar to the methods for collecting microvascular endothelial cells in adipose tissue.

Moving forward, the heterogeneous mixture of adipose-derived cells (i.e., the PLA) may then be obtained from the disaggregated tissue fragments by reducing the presence of mature adipocytes. Particularly, separation of the cells from a suspension of the PLA may be achieved with any suitable process including centrifugation, buoyant density sedimentation, elutriation, and the like. For example, when a centrifuge is utilized, the centrifugation process may form a “pellet,” which may optionally be resuspended with a buffered physiologic solution. In one exemplary embodiment, the isolated mixture of cells may then be passed to a mixing container to mix the cells with another component, such as adipose tissue. In this embodiment, the adipose tissue may function as a natural scaffold. In another exemplary embodiment, the isolated cells may be passed to a chamber where the cells are adhered to an element such as a graft that may be implanted within a patient.

As should be obvious to those skilled in the art based upon the foregoing discussion, certain embodiments of the invention may be directed to methods (and compositions formed by methods) of fully disaggregating the adipose tissue to separate the active cells from the mature adipocytes and connective tissue, while other embodiments of the invention may be directed to methods (and compositions formed by methods) in which the adipose tissue is only partially disaggregated.

As will be appreciated by those skilled in the art, the adipose cells that have been processed and concentrated as described above may be administered to the patient without further processing, or they may alternatively be administered to the patient after being combined with other tissues, cells, or devices in a mixing container or chamber as previously mentioned. In one exemplary embodiment, the isolated cells or mixture of cells may be combined with a portion of adipose tissue that has not been similarly processed. Thus, in accordance with the present invention, a composition comprising adipose tissue with an enhanced concentration of active cells may be administered to the patient for treatment. The concentration of active cells in the composition is customizable and dependent upon the amount of unprocessed adipose tissue that is utilized. Alternatively, the active cells may be combined with other types of cells, tissue, tissue fragments, demineralized bone, growth factors such as insulin or drugs such as members of the thiaglitazone family, biologically active or inert compounds, resorbable plastic scaffolds, or other additives intended to enhance the delivery, efficacy, tolerability, or function of the population.

In certain embodiments of the present invention, the adipose-derived cells may be administered directly into the patient. In other words, the active cell population (e.g., the stromal vascular fraction) may be administered to the patient without being removed from the system or exposed to the external environment of the system. As will be appreciated by those skilled in the art, providing a closed system reduces the chances that contamination will occur. Obviously when a closed system is used the adipose cells are not processed for culturing or cryopreserved. Rather, the cells are harvested for immediate use in the patient.

Regardless of whether an “open” or “closed” system is used, or whether the concentrated cells are administered immediately or are preserved for later use, the cells may be loaded into any suitable delivery system, such as a syringe, for administering into the recipient. The cells may be administered by, for example, subcutaneous, intravenous, intramuscular, or intraperitoneal techniques. In other words, it is contemplated that the cells may be delivered to the patient by any means known to those of ordinary skill in the art, including injection into blood vessels for systemic or local delivery, into tissues, into the dermis, into tissue space, or into any other suitable location.

Now that exemplary systems and methods for administering isolated adipose-derived cell populations in various forms have been described, several therapeutic procedures in accordance with the present invention utilizing these cell populations will be described.

Particularly, the inventors have found that cell based therapies have the potential to treat many pelvic health disorders such as urinary incontinence, erectile dysfunction, and bladder disorders including interstitial cystitis. As a treatment modality, adipose-derived cell populations may contribute to the regeneration and/or repair of critical structures in the pelvic floor. As appreciated by those skilled in the art, an important aspect of a cell's ability to provide functional support in any environment is its ability to be retained in that environment. In accordance with the present invention, it has been found that one method to increase retainment is to provide an attachment surface for adipose-derived cells. As such, the addition of a scaffold material to an isolated population of adipose-derived cells that has been harvested in a manner such as that described in detail above prior to injection may contribute to the overall effectiveness of the therapy.

Generally speaking, and in accordance with the present invention, the use of adipose tissue as a source of therapeutic cells provides the ability to obtain a natural scaffold for the cells. It has been shown that autologous fat alone has a limited life within the human body. The conjectured cause of the phenomenon is the lack of vascularization of the tissue to sustain the newly moved tissue. However, this weakness may be mitigated by mixing the therapeutic cells with unprocessed adipose tissue, which provides the capacity to aid in vascularization. It has also been observed that cells tend to migrate to sites of damage. Using adipose tissue as an autologous scaffolding has been found to mitigate such migration and maintain the therapeutic cells in the intended location within the patient's body.

Thus, in one exemplary embodiment, adipose tissue may be removed from a patient and divided into two parts. The first part may be processed via any suitable cell isolation process so as to obtain a heterogeneous population of adipose-derived cells. The second part may be left “undigested” so that it may be used as a scaffold for the adipose-derived cells. Prior to injection or placement into the patient, the adipose tissue and the adipose-derived cells may be mixed together in any suitable ratio including, but not limited to, 1:1, 1:2, 1:3, 1:4 or any ratio falling therebetween. The tissue/cell composition may then be injected into the urethra to treat urinary incontinence, the corpus cavernosa to treat erectile dysfunction, the bladder to treat interstitial cystitis, or any other pelvic floor structure in need of tissue regeneration or repair.

With further regard to urinary incontinence, the following is one exemplary method that may be used to treat the disorder in accordance with the present invention. First, a 1:1 composition formed from autologous fat and a heterogeneous cell mixture derived from adipose tissue may be created as previously described. As described herein, the precise ratio may be anywhere from approximately 1:1 to approximately 1:4. Then, a first set of periurethral injections may be initiated up to the bladder neck of the patient. These injections may be made under the guidance of any suitable imaging device, such as a cystoscope, in order to confirm urethral closure. Because the ratio of the composition being injected consists of an equal part of adipose tissue, it will have an increased bulking effect and will be of greater volume than a cell mixture that lacks the added adipose tissue. As will be appreciated by those skilled in the art, the volume injected may cause an inflammatory effect that could hasten the remodeling of the tissue, thereby improving the long term outcome.

In addition to the periurethral injections, a second set of injections could also be made through, for example, the anterior vaginal wall along the proximal two-thirds of the urethra just short of the bladder neck. Exemplary locations for these injections may include submucosa, sphincter urethrae, external sphincter (compressor urethrae and urethrovaginalis), urogenital hiatus, and puborectalis (or levator ani). The second set of injections, while potentially more numerous, may preferably include only the heterogeneous mixture of cells instead of the composition formed with adipose tissue described above. Once injected, the heterogeneous mixture of cells may begin regenerating the tissue through cell communication, vascularization, and cell guidance. Ideally, the projected time for the functionality of these cells may coincide with the time associated with the degradation of injected adipose tissue as discussed above. However this degradation may be limited by the presence of cells within the tissue that retain regenerative capabilities.

Because the cells in the heterogeneous mixture of cells produced in accordance with the present invention are not cultured, and are therefore not outside of the body for an extended period of time or preserved with chemicals or agents, the entire process is a natural process. Further, apoptosis can occur spontaneously if functionality is not provided. This process would not result in any harm to the subject as cells continuously proceed through life and death stages. However, cells responding to signals within the region will differentiate to offer appropriate functionality as needed.

With regard to erectile dysfunction, causality may be neurogenic or vasculogenic in nature. Further, erectile dysfunction may be caused by injury or trauma such as radical prostatectomy. The nature of the cell solution described herein having cellular precursors for vasculature and a population of minimally differentiated stem cells suggest a beneficial impact on neurogenic and vasculogenic repair. These cells (or cells in combination with adipose tissue) may be injected into the corpora cavernosa and held within the penis with a temporary tourniquet for a time period of from 5 to 15 minutes. This would allow the cells to attach to native tissue or remain with the adipose or other scaffold for long term retention in the penis. The function of the retained cells would be to help, guide, or provide additional tools for neurogenic and vascular repair. Sodding of cells into the pelvic region during a pelvic procedure such as radical prostatectomy may also be utilized. Such a technique may have a direct effect on the neural injury by restoring the nerve at the site of injury to its original level of function and providing additional cells to increase local vascularization for more rapid healing. In yet a further technique, the novel concentrated cell populations may also be “pasted” into the empty space of the prostate using a scaffold similar to a fibrin sealant for local cellular retention.

With further regard to bladder disorders, the present inventors have discovered that cells harvested from adipose tissue may offer significant advantage to people suffering with bladder conditions including interstitial cystitis, bladder pain, and overactive bladder. Historically, bladder disorders have been treated with non-natural substances such as BOTOX®. As appreciated by those skilled in the art, BOTOX® is a damaging toxin capable of reducing various symptoms of bladder disorders though neuro and muscular degeneration.

In one exemplary embodiment, mesenchymal cells may be collected from adipose tissues from a patient's abdominal region, concentrated through a process of tissue digestion and centrifugation as previously described, and positioned back within the patient in their isolated state, in a composition, or in conjunction with a graft or the like. Thus, in contrast to damaging tissue through the use of a toxin, the present invention may utilize concentrated autologous cells for treatment. In accordance with one exemplary method of delivering the cells to the patient, the concentrated cell population may be injected via a needle or similar device under the guidance of a cystoscope. For instance, about 30 small injections may be made across the surface of the bladder into the submucosal tissue and deep muscle tissue. As will be appreciated by those skilled in the art, this solution may offer the local environment an autologous cellular response with the potential of healing the damage to the urothelium, muscles, or nerves. The diverse population of injected cells derived from the adipose tissue has a vasculogenic capability and retains the ability to differentiate into different cell types. The vascular potential may help to provide the nutrients and cell factors needed for the tissue to heal. Furthermore, the minimally differentiated cells may add to a limited population and bring other cells to hasten healing through cytokine signaling and other cell-to-cell interactions. For example, this healing may occur within the urothelium to treat ulcers or weaknesses within the tissue, nerve tissues to provide cellular needs for the treatment of neurogenic disorders, or other local tissues to improve/restore function or eliminate discomfort.

Those skilled in the art will appreciate that although reference was made to the collection of adipose cells from the abdominal region in the example set forth above, the cells may alternatively be collected from any other source of adipose tissue without departing from the intended scope of the present invention. Furthermore, the adipose cells may be concentrated by means other than centrifugation that are well known to those skilled in the art.

### EXAMPLE

### Treatment of Erectile Dysfunction in Rats Using Cell Based Therapy

This example describes the treatment of erectile dysfunction with a heterogeneous mixture of adipose-derived cells isolated from the abdominal region of normal adult Sprague Dawley rats. The heterogeneous mixture of cells includes but is not limited to adipose-derived stem cells, endothelial cells and their precursors, and smooth muscle cells and their precursors. Each of the rats underwent with nerve crush injuries to the cavernous nerves.

Cell Collection and Concentration Process:

Adipose tissue was removed from a rat and washed in sterile saline. A collagenase solution was mixed with the adipose tissue and incubated at 37° C. for 30 minutes with manual shaking. After incubation, the cells were washed by centrifugation twice and filtered. The centrifuge drive mechanism was then oscillated to allow the collagenase to digest the adipose tissue. On average, the collagenase digests the adipose tissue in approximately 30 minutes. A cell count was conducted on the resulting heterogeneous mixture of cells and administered to the rat or mixed with adipose tissue and then administered to the rat.

Results:

The rats were randomly divided into three different groups. In the first group of rats, a heterogeneous mixture of cells alone (about two million cells suspended in saline solution) was injected into the corpus cavernosa of each rat in the group. In the second group of rats, a heterogeneous mixture of cells (about two million cells suspended in saline solution) was combined with washed, mechanically digested adipose tissue in an approximately 1:1 ratio and injected into the corpus cavernosa of each rat in the group. The adipose tissue was mechanically digested by chopping the tissue with a scalpel to form an injectable slurry. Finally, in the third group, saline solution alone was injected into the corpus cavernosa of each of the rats in the group. After three months, the test results were as follows: (1) in the first group, 5/14 or about 35.7% of the rats had a positive response to electrical stimulation; (2) in the second group, 10/15 or about 66.7% of the rats had a positive response to electrical stimulation; and (3) in the third group, 1/15 or about 6.7% of the rats had a positive response to electrical stimulation. A positive test response was defined as a ratio of intracavernosal pressure to mean arterial pressure greater than 0.4 cm H₂O. The results suggest that the addition of a scaffold material such as adipose tissue increases the efficacy of the cell based therapy.

Although the present invention has been described with reference to preferred embodiments, workers skilled in the art will recognize that changes may be made in form and detail without departing from the spirit and scope of the invention.

What is claimed is:

1. A method for treating a pelvic floor disease comprising:
removing adipose tissue from a patient;
processing a first portion of the adipose tissue to obtain a heterogeneous mixture of cells that includes adipose-derived stem cells;
combining the heterogeneous mixture of cells with a second, unprocessed portion of the adipose tissue in a ratio of from approximately 1:1 to 1:4 to produce a cell composition, wherein the second portion of the adipose tissue is structured to provide a natural scaffold; and
administering the cell composition into the patient to treat a pelvic floor disease.

2. The method of claim 1 wherein administering the cell composition into the patient is done by injection to derive a therapeutic or structural benefit to the patient.

3. The method of claim 2 wherein the cell composition is injected into the corpus cavernosa.

4. The method of claim 2 wherein the cell composition is injected directly into the sphincter or into spaces around the urethra.

5. The method of claim 2 wherein the cell composition is injected directly into the bladder.

6. The method of claim 2 wherein the cell composition is administered to the patient by periurethral injection up to the bladder neck of the patient to induce an increased bulking effect.

7. The method of claim 6 wherein the cell composition is administered to the patient by a second set of injections in the anterior vaginal wall along the proximal two-thirds of the urethra just short of the bladder neck including the submucosa, sphincter urethrae, external sphincter, urogenital hiatus, puborectalis and levator ani.

8. The method of claim 1 wherein said processing further includes (i) washing the adipose tissue, (ii) centrifuging the adipose tissue, (iii) enzymatically dissociating the adipose tissue using an enzyme selected from the group consisting of trypsin, collagenase, dispase or combinations thereof to produce a heterogeneous concentrated stem cell population including myoblasts, fibroblasts, nerve cells, endothelial cells and adipocytes; (iv) washing the concentrated stem cell population in a sterile balanced salt solution; and (v) centrifuging the concentrated stem cell population.

9. The method of claim 1 wherein combining the heterogeneous mixture of cells with a second, unprocessed portion of the adipose tissue is done in a ratio of approximately 1:1 to produce a cell composition.

10. The method of claim 1 wherein combining the heterogeneous mixture of cells with a second, unprocessed portion of the adipose tissue in a ratio of approximately 1:2 to produce a cell composition.

11. The method of claim 1 wherein combining the heterogeneous mixture of cells with a second, unprocessed portion of the adipose tissue is done in a ratio of approximately 1:4 to produce a cell composition.

12. Use of a cell composition containing a first portion of unprocessed adipose tissue and a second portion of an isolated adipose-derived, stem-cell population to treat urinary incontinence, pelvic prolapse or erectile dysfunction.

13. Use of a cell composition in accordance with claim 12 wherein the adipose-derived, stem cell population is obtained by removing adipose tissue from a patient and processing the adipose tissue to obtain a heterogeneous mixture of cells that includes adipose-derived stem cells.

14. Use of a cell composition in accordance with claim 12 wherein the first portion of unprocessed adipose tissue and the second portion of an adipose-derived, stem-cell population is mixed in a ratio of from approximately 1:1 to 1:4 prior to said use.

15. Use of a cell composition in accordance with claim 12 wherein the isolated adipose-derived, stem-cell population in the second portion contains a heterogeneous mixture of cells including from about 1% to 2% stem cells.

16. Use of a cell composition in accordance with claim 13 wherein said processing includes (i) washing the adipose tissue, (ii) centrifuging the adipose tissue, (iii) enzymatically dissociating the adipose tissue using an enzyme selected from the group consisting of trypsin, collagenase, dispase or combinations thereof to produce a heterogeneous concentrated stem cell population including myoblasts, fibroblasts, nerve cells, endothelial cells and adipocytes; (iv) washing the concentrated stem cell population in a sterile balanced salt solution; and (v) centrifuging the concentrated stem cell population.

17. A cell composition containing a first portion of unprocessed adipose tissue and a second portion of an isolated adipose-derived, stem-cell population to treat urinary incontinence, pelvic prolapse or erectile dysfunction.

18. The cell composition of claim 12 wherein the adipose-derived, stem cell population is obtained by removing adipose tissue from a patient and processing the adipose tissue to obtain a heterogeneous mixture of cells that includes adipose-derived stem cells.

19. The cell composition of claim 12 wherein the first portion of unprocessed adipose tissue and the second portion of an adipose-derived, stem-cell population is mixed in a ratio of from approximately 1:1 to 1:4 prior to said use.

20. The cell composition of claim 12 wherein the isolated adipose-derived, stem-cell population in the second portion contains a heterogeneous mixture of cells including from about 1% to 2% stem cells.

21. The cell composition of claim 13 wherein said processing includes (i) washing the adipose tissue, (ii) centrifuging the adipose tissue, (iii) enzymatically dissociating the adipose tissue using an enzyme selected from the group consisting of trypsin, collagenase, dispase or combinations thereof to produce a heterogeneous concentrated stem cell population including myoblasts, fibroblasts, nerve cells, endothelial cells and adipocytes; (iv) washing the concentrated stem cell population in a sterile balanced salt solution; and (v) centrifuging the concentrated stem cell population.
